(19) United States
(12) Patent Application Publication
(10) Pub. No.: US 2009/0182423 A1
(43) Pub. Date: Jul. 16, 2009

(54) CRYSTALLINE LENS CAPSULAR SUPPORT DEVICE FOR USE IN A SURGICAL PROCEDURE ON A HUMAN EYE

(76) Inventor: Lei Zheng, Fort Lee, NJ (US)

Correspondence Address: LEI ZHENG, 234A WASHINGTON AVE., FORT LEE, NJ, 07024, US

(21) Appl. No.: 12/015,454

(22) Filed: Jan. 16, 2008

(51) Int. Cl. A61F 2/16

(52) U.S. Cl. 623/6.39

(57) ABSTRACT

Presented is a device for use in a surgical procedure on an eye. The device includes a thin layer of membrane made with biocompatible materials, and shaped with curved posterior portion, curved anterior portion, like the shape of a human crystalline lens capsule bag. The front portion has a circled opening. A number of closed haptics could attach to the equator portion of the device. This device is to support the lens capsule after cataract surgery, or any other typed of eye surgery.

### FIELD

The disclosed subject matter relates to lens capsular support for use in a surgical procedure on an eye.

### BACKGROUND

Phacoemulsification with in-the-bag intraocular lens (IOL) implantation has been the standard of care for cataract surgery. Secure fixation of the IOL in-the-bag is critical for a good outcome, but does not guarantee an uncomplicated postoperative course. Posterior capsule opacification (PCO), capsule shrinkage, posterior capsule crinkle, vitreous detachment, and cystoid macular edema (CME) are some well-known complications of state-of the-art cataract surgery. Decentration or dislocation of a posterior chamber IOL can occur after cataract surgery. Dislocation of an intraocular lens (IOL) is reported with increasing frequency in recent years. Pseudoexfoliation, uveitis, myopia, and other diseases associated with progressive zonular weakening and capsular contraction are the predisposing conditions.

Pars plana vitrectomy is commonly used in the management of posterior pole pathology including retinal detachment, macular hole surgery, and epiretinal membrane removal. Cataract formation is common after vitrectomy in phakic eyes. Cataract surgery in postvitrectomized eyes is challenging because of the loss of vitreous support, weakened zonules, posterior capsule plaques, and weakened posterior capsules.

Posterior capsule (PC) rupture during phacoemulsification cataract surgery remains an important complication because it may lead to poor visual outcome. Vitreous loss is associated with various complications including cystoid macular edema, glaucoma, retinal detachment, and infective endophthalmitis.

Cataract extraction is the most commonly performed surgery in the United States with more than 2 millions phacoemulsification procedures in recent years. This number is increasing due to the increased population and life span. Capsular rupture is noted to occur in about 0.9% of cases. Although there are a variety of lens options which exist for patients who lack adequate capsular support for a bag-fixated or sulcus-fixated posterior chamber, most surgeons consider the flexible open-loop AC IOL and the trans-sclerally sutured PC IOL to be the most acceptable alternatives. But none of these methods are considered safe, and both of these methods have considerable complications. Trans-scalerally sutured PC IOL is considered better choice over AC IOL, but is very difficulty to perform and may cause considerable trauma to the eye.

Human crystalline lens is approximately from 4 to 5 mm in thickness and 8 to 10 mm in diameter. PC IOL is ranged approximately from 0.7 to 0.9 mm in thickness and 5.5 to 6 mm in diameter. After cataract surgery with PC IOL, the anterior chamber depth will increase about 1.0 mm. It means that there is about 2 to 3 mm space left between the back of IOL and the front of the anterior vitreous. This space may become the major reasons for vitreous detachment and other post operation disorders.

### SUMMARY

In one aspect, the disclosed subject matter relates to a lens capsule support device for use in a surgical procedure on an eye. The device is as the following:
1. The lens capsule support device is a thin layer of biocompatible plastic membrane, such as PMMA.
2. The shape of the Lens capsule support device is constructed with curved posterior portion, equator portion and curved short anterior portion (FIGS. 2 and 3).
3. Lens capsule support device can attach 3 to 8 closed haptics. (FIGS. 4, 5, 6, 7, and 8).
4. The equator portion of the Lens capsule support device can be smoothly curved (FIG. 4), or flat (FIG. 5).
5. The thickness of the membrane is best to be ranged from 3 μm to 20 μm, or more if needed. The distance from the center of the posterior portion to the center of the anterior portion is best to be 3.5 to 4 mm. The diameter (distance from equator to opposite equator) of the Lens capsule support device is best to be 9 to 11 mm, but may vary based on the need (FIGS. 2, 3). With 3 to 8 closed haptics, the diameter the Lens capsule support device is best to be around 7 to 9 mm, and the total diameter (with haptics) is best to be 11 to 12 mm (FIGS. 4, 5, 6). If the Lens capsule support device intended to be rested on ciliary body, the total diameter (with haptics) is best to be 12 to 14 mm (FIG. 14). All the scales mentioned above are referenced number; they can be more or less, based on the need.

6. Lens capsule support device is a soft biocompatible plastic membrane, so it can be inserted through a small incision into the lens capsule bag after phacoemulsification, such as through 2.6 mm, or 3 mm cornea incision.
7. Lens capsule support device should be hard enough to resist the forwarding force from vitreous. And it should be able to support the lens capsule in its original shape (FIGS. 9, 10, 11, 12).

In another aspect, the disclosed subject matter involves a method of performing a surgical procedure on an eye. The method includes making an incision in the cornea of an eye, and inserting into the eye through the incision, and supporting the lens capsule, or functioning as lens capsule with, a device as described above.

### BRIEF DESCRIPTION OF THE DRAWINGS

In the drawings, like reference characters refer to the same parts throughout the different views. Also, the drawings are not necessarily drawn to scale. The scales here mentioned are referenced number; they can be more or less, based on the need.

FIG. 1 is a human crystalline lens. It has anterior portion, posterior portion and equator portion.

FIG. 2 is the cut of side view of the lens capsule support device.

FIG. 3 is the front view of the lens capsule support device. The inner circle is the edge of the front opening. The outer circle is the equator portion.

FIG. 4 is the cut side view of the lens capsule support device with closed haptics.

FIG. 5 is the cut side view of the lens capsule support device with flat equator.

FIG. 6 is the front view of the lens capsule support device with 6 closed haptics.

FIG. 7 is a magnified cut side view of the lens capsule support device shows the attachment of the haptic.

FIG. 8 is a magnified cut side view of the lens capsule support device with flat equator shows the attachment of the haptic.

FIG. 9 is the lens capsule support device inside the lens capsule bag after phacoemulsification and support the lens capsule in its original shape.

FIG. 10 shows the PC IOL was implanted inside the lens capsule support device.

FIG. 11 shows the lens capsule support device with haptics inside the lens capsule supporting the lens capsule in its original shape.

FIG. 12 shows the PC IOL was implanted inside the lens capsule support device with closed haptics.

FIG. 13 shows the implanted PC IOL without lens capsule support device has lens capsule deformation and posterior capsule crinkle.

FIG. 14 shows the lens capsule support device with haptics rested on the ciliary body.

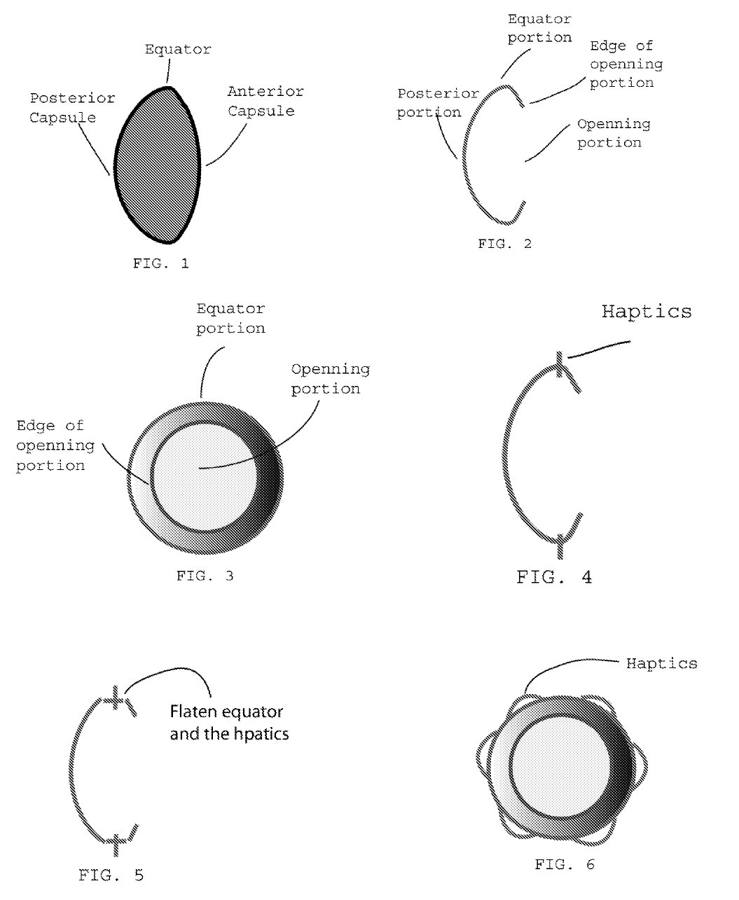

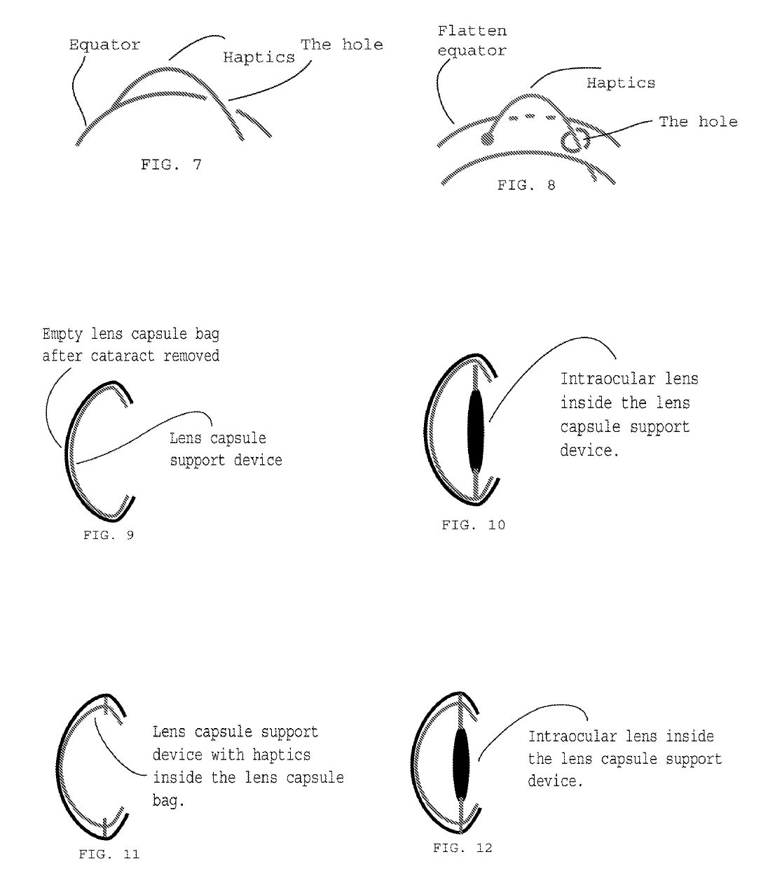

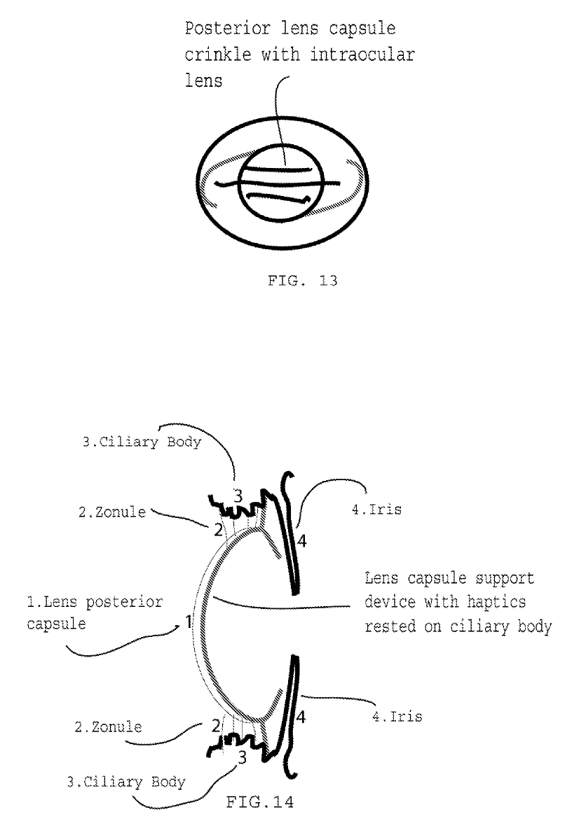

### BENEFITS WITH LENS CAPSULE SUPPORT DEVICE

1. The lens capsule support device could be able to prevent the shrinkage, deformation, and crinkle of the posterior capsule after cataract surgery with PC IOL and keep the lens capsule in its original shape.

2. The lens capsule support device could be able to prevent the decentration and dislocation of a posterior chamber IOL.

2. The lens capsule support device could be able to prevent the forward moving of the vitreous, so it may be able to prevent, or reduce the chance of vitreous detachment, cystoids macular edema and other retina disorders after cataract surgery

3. The lens capsule support device may be able to prevent PCO by increasing the force and tight contact between the back of the Lens capsule support device and the front of the vitreous.

4. The lens capsule support device may be able to facilitate the implantation of PC IOL when the posterior capsule has a tear, rupture or the weakened zonules.

What is claimed is:

1. A device for use in a surgical procedure on an eye, comprising: a thin layer of biocompatible membrane with posterior portion, equator portion and anterior portion (with an opening).

2. The device according to claim 1, wherein the posterior portion is curved.

3. The device according to claim 1, wherein the anterior portion with an opening is curved.

4. The device according to claim 1, wherein the equator portion could be curved, or flat.

5. The device according to claim 1, wherein the equator portion can add closed haptics.
